(19) United States
(12) Patent Application Publication
(10) Pub. No.: US 2025/0312579 A1
(43) Pub. Date: Oct. 9, 2025

(54) APPARATUS FOR SHUNTING CEREBROSPINAL FLUID HAVING AN ARTIFICIAL FONTANELLE

(71) Applicant: Gary MAGRAM, Corona Del Mar, CA (US)

(72) Inventor: Gary MAGRAM, Corona Del Mar, CA (US)

(21) Appl. No.: 18/627,760

(22) Filed: Apr. 5, 2024

(51) Int. Cl. A61M 27/00; A61M 25/00; A61M 39/10; A61M 39/24

(52) U.S. Cl. CPC A61M 27/006; A61M 25/0026; A61M 25/007; A61M 39/105; A61M 39/24; A61M 2027/004; A61M 2039/248; A61M 2205/3331; A61M 2205/3379; A61M 2210/0687; A61M 2210/0693

(57) ABSTRACT

Apparatus for shunting cerebrospinal fluid comprising a ventricular catheter coupled to an artificial fontanelle and a peritoneal catheter. The ventricular catheter may be coupled to the artificial fontanelle and peritoneal catheter via a three-way coupler or the ventricular catheter may be coupled to the peritoneal catheter through the artificial fontanelle. The cerebrospinal fluid flow resistance is lower between the ventricular catheter and the artificial fontanelle than the cerebrospinal fluid flow between the artificial fontanelle and the peritoneal catheter.

### BACKGROUND

### Field

Embodiments of the present invention generally relate to cerebrospinal fluid shunts and, in particular, to an apparatus for shunting cerebrospinal fluid having an artificial fontanelle that may be used to treat hydrocephalus.

### Description of the Related Art

In a normal mammal, cerebrospinal fluid (CSF) is created within ventricles of the brain and flows into and out from the spinal column. CSF flow is affected by a combination of forces acting upon fluids within the body including arterial pulsations, body movement, body orientation (i.e., standing, sitting, or prone), atmospheric pressure, respiration, diaphragmatic movement, abdominal motion, and the like. When the flow of CSF is constrained, a condition known as hydrocephalus occurs which results in CSF pressure increasing in the head. Untreated hydrocephalus may lead to brain damage and/or death.

Currently, hydrocephalus is treated with a CSF shunt that facilitates channeling CSF from the head or spine into another portion of the body (e.g., vein or peritoneal cavity) where the CSF is absorbed. A typical shunt comprises a ventricular catheter, a subgaleal catheter, a one-way pressure activated valve, and a distal subcutaneous peritoneal catheter. Some shunts also include a reservoir preceding the valve and an anti-siphon regulatory device after the valve. The ventricular catheter has its proximal end placed in a ventricle in the brain and a distal end that is connected to the reservoir or valve. The reservoir, valve and anti-siphon device may be connected in series and placed under the skin, typically, on the back or side of the skull. The peritoneal catheter has a proximal end that is connected to the anti-siphon device and a distal end extending to the site of CSF absorption (into a vein, the peritoneal cavity, or pleural cavity).

In operation, as CSF is produced in the ventricles and increases the pressure in the ventricles, the valve opens when the pressure exceeds the pressure threshold of the valve, the catheter resistance, the intraperitoneal pressure, plus the hydrostatic pressure difference. When the valve opens, CSF flows through the shunt to the peritoneal cavity or vein where the CSF is absorbed by the body. The reservoir is provided as a CSF access device and the anti-siphon device ensure that a siphon effect does not maintain the valve in on open position and drain all the CSF from the brain and spine (the reservoirs are, in general, only deformable by significant external pressure, they are otherwise non-compliant to physiological pressures).

A major problem with a shunt is that it is purely a pressure-based device that forms an alternate CSF path. Consequently, the typical shunt attenuates the other forces that effect CSF flow. Thus, the complex flow path for CSF is bypassed by the shunt and results in a variety of medical conditions such as intracranial hypotension and slit ventricles.

Therefore, there is a need for apparatus for shunting CSF that more closely mimics the complex movement of CSF within the body.

### SUMMARY

Apparatus for shunting cerebrospinal fluid having an artificial fontanelle is provided substantially as shown in and/or described in connection with at least one of the figures, as set forth more completely in the claims.

Various features and advantages of the present disclosure may be appreciated from a review of the following detailed description of the present disclosure, along with the accompanying figures in which like reference numerals refer to like parts throughout.

### BRIEF DESCRIPTION OF THE DRAWINGS

So that the manner in which the various features of the present invention can be understood in detail, a particular description of the invention, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

FIG. 1 depicts a schematic of a person with a cerebrospinal fluid shunt having an artificial fontanelle in accordance with at least one embodiment of the invention;

FIG. 2 depicts a top view of a person's head with a cerebrospinal fluid shunt having an artificial fontanelle in accordance with at least one embodiment of the invention;

FIG. 3 depicts a horizontal cross-sectional view of a three-way connector of the shunt of FIG. 2 in accordance with at least one embodiment of the invention;

FIG. 4 depicts a vertical cross-sectional view of the three-way connector of the shunt of FIG. 2 in accordance with at least one embodiment of the invention;

FIG. 5 depicts a horizontal cross-sectional view of a three-way connector with a non-compliant reservoir in accordance with at least one alternative embodiment of the invention;

FIG. 6 depicts a horizontal cross-sectional view of an artificial fontanelle in accordance with at least one embodiment of the invention;

FIG. 7 depicts a vertical cross-sectional view of an artificial fontanelle along line 7-7 in FIG. 6 in accordance with at least one embodiment of the invention;

FIG. 8 depicts a horizontal cross-sectional view of an artificial fontanelle in accordance with at least one alternative embodiment of the invention;

FIG. 9 depicts a vertical cross-sectional view of an artificial fontanelle along line 9-9 in FIG. 8 in accordance with at least one alternative embodiment of the invention;

FIG. 10 depicts a horizontal cross-sectional view of an artificial fontanelle in accordance with at least one alternative embodiment of the invention;

FIG. 11 depicts a vertical cross-sectional view of an artificial fontanelle in accordance with at least one alternative embodiment of the invention;

FIG. 12 depicts a perspective view of a ventricular catheter for use with an artificial fontanelle in accordance with at least one embodiment of the invention;

FIG. 13 depicts a vertical cross-sectional view of the ventricular catheter of FIG. 12 in accordance with at least one embodiment of the invention;

FIG. 14 depicts a vertical cross-sectional view of a vertical-horizontal sensing ball valve in accordance with at least one embodiment of the invention;

FIG. 15 depicts a horizontal cross-sectional view of the vertical-horizontal sensing ball valve along line 15-15 of FIG. 14 in accordance with at least one embodiment of the invention; and

FIG. 16 depicts a horizontal cross-sectional view of a dual lumen catheter along line 16-16 of FIG. 14 in accordance with at least one embodiment of the invention.

### DETAILED DESCRIPTION

Embodiments of the present invention comprise a cerebrospinal fluid (CSF) shunt having an artificial fontanelle. Such a shunt is improved over currently available shunts by including the artificial fontanelle that allows the shunt operation to more closely mimics the complex movement of CSF within the body. The artificial fontanelle forms a subgaleal flexible reservoir for CSF positioned on the top of the skull which fills with CSF and permits CSF to flow into and out of the ventricles of the brain.

In one embodiment, the shunt comprises a ventricular catheter (also known as a proximal catheter), a three-way connector, an artificial fontanelle, a one-way pressure valve and a distal (peritoneal) catheter. The ventricular catheter has a first end inserted into a ventricle of the brain through a bore hole in the skull and a second end coupled to a first port of the three-way connector. A second port of the connector is connected to the artificial fontanelle and a third port is connected, via a catheter, to the one-way pressure valve. The valve is also connected to the distal catheter that terminates in the peritoneal cavity. The first port, second port, ventricular catheter and inlet to the artificial fontanelle have a diameter that is larger than the third port and the catheter coupling the third port to the valve. In this manner, CSF readily flows between the ventricle and the artificial fontanelle in a bidirectional manner via the larger diameter connection. CSF flows through the valve and peritoneal catheter when the differential pressure across the valve is sufficient to open the valve. In some embodiments, the shunt may further include an anti-siphon device located in the distal catheter near the valve. In other embodiments, the valve may be replaced with a vertical-horizontal sensing ball valve that controls the amount of CSF flowing in the peritoneal catheter depending upon whether a person is prone (horizontal) or sitting/standing (vertical).

In other embodiments, the shunt may not use a three-way connector and, instead, the artificial fontanelle may be connected directly to the large diameter ventricular catheter via a large diameter first port of the fontanelle. In such an embodiment, the artificial fontanelle comprises a second port having a smaller diameter than the first port. The second port is coupled to the one-way valve or vertical-horizontal sensing ball valve. In all embodiments, a shunt having of an artificial fontanelle facilitates a more natural flow of CSF into and out of the ventricle than a traditional shunt.

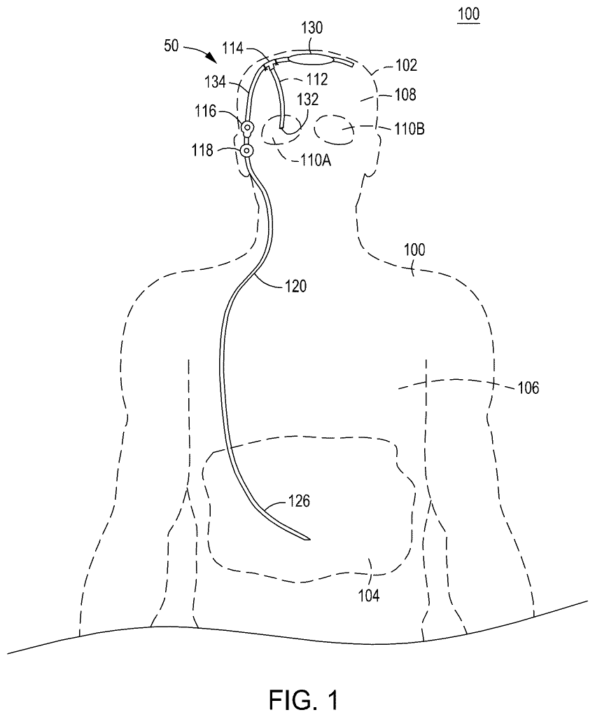

FIG. 1 depicts a schematic of a person 100 with a cerebrospinal fluid (CSF) shunt 50 having an artificial fontanelle 130 in accordance with at least one embodiment of the invention. The person 100 has a head 102 and torso 106. The head contains a brain 108 located in the head 102 and an abdomen 104 located in the torso 106. The shunt 50 extends from a ventricle 110A or 110B in the brain 102 to the abdomen 104 such that excess CSF can flow from the head 102 to the abdomen 104. In one embodiment, the shunt 50 comprises a ventricular catheter 112, a three-way connector 114, a one-way valve 116, an optional siphon regulatory device 118, and a subcutaneous peritoneal catheter 120.

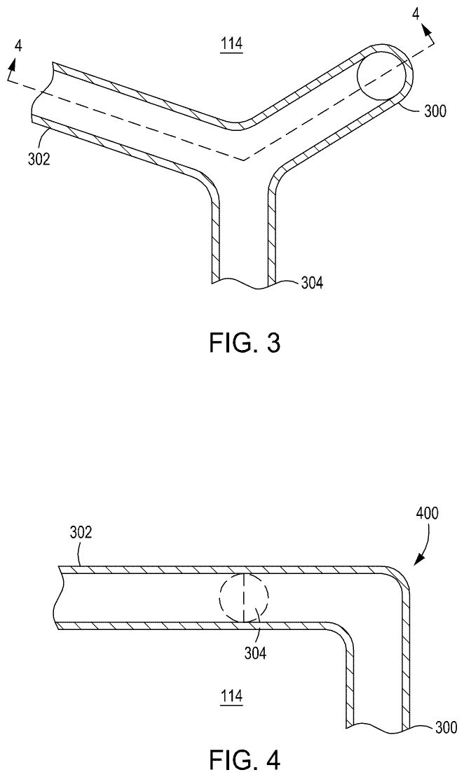

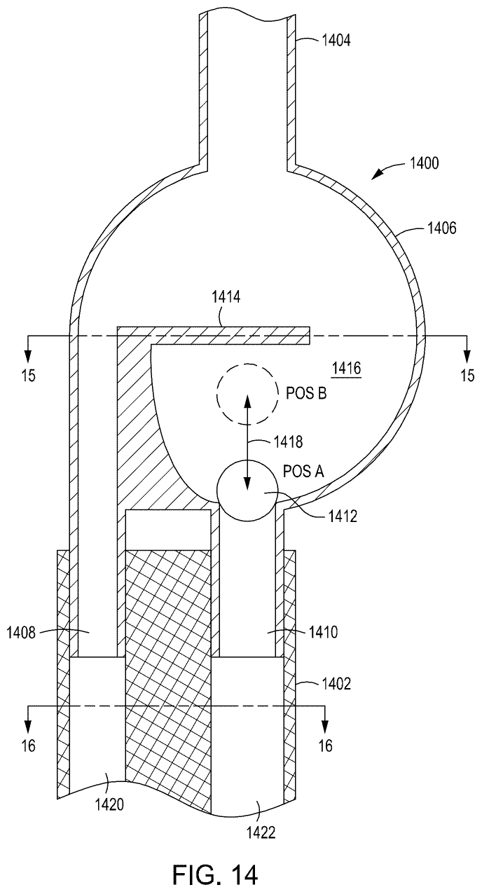

The ventricular catheter 112 has a proximal end 132 that is positioned in one of the ventricles 110A or 110B. The ventricular catheter 112 extends outside the skull though a burr hole and is coupled to a coupling or subgaleal catheter 134. The coupling catheter 134 is routed subcutaneously to the three-way connector 114. The three-way connector 114 couples the coupling catheter 134 to the artificial fontanelle 130 and the ventricular catheter 112. The connector 114 is described in detail with respect to FIGS. 3, 4 and 5 below. The combination of the valve 116, an optional anti-siphon device 118 and the peritoneal catheter 120 form a conventional shunt assembly that carries CSF from the valve 116 to portion 126 of the peritoneal catheter 120 located in the abdomen 104. The portion of the catheter 120 typically are located subcutaneously along the back of the head 102. A central portion of the catheter 120 is routed along the chest of the person 100. The distal end 126 of the catheter 120 terminates in the peritoneal cavity of the abdomen 104. In an alternative embodiment, the valve 116 may be a vertical-horizontal sensing ball valve as described in detail below with respect to FIGS. 14, 15, and 16.

The artificial fontanelle 130 forms compliant, flexible CSF reservoir having a volume of between 0.8 mL and 5 mL. In one embodiment, the volume is between 2 mL and 5 mL. To facilitate CSF flow between the ventricle 110A and the artificial fontanelle 130, a large diameter ventricular catheter 112 is used. The inner diameter of the catheter 112 and the first and second ports of the connector 114 is about 2 mm. The ports are tubes defining a lumen with an inner diameter of about 2 mm. While the inner diameter of the third port of the connector 114 and the remaining components (134, 116, 118, and 120) of the shunt 50 is about 0.5 mm. In this manner, the CSF flow path between the ventricle 110A and the artificial fontanelle 130 has low flow resistance and the CSF flow path through the catheter 134 has high flow resistance. Consequently, bidirectional CSF flow between the ventricle 110A and the artificial fontanelle 130 is hydrodynamically preferred and mimics the normal flow of CSF in the body.

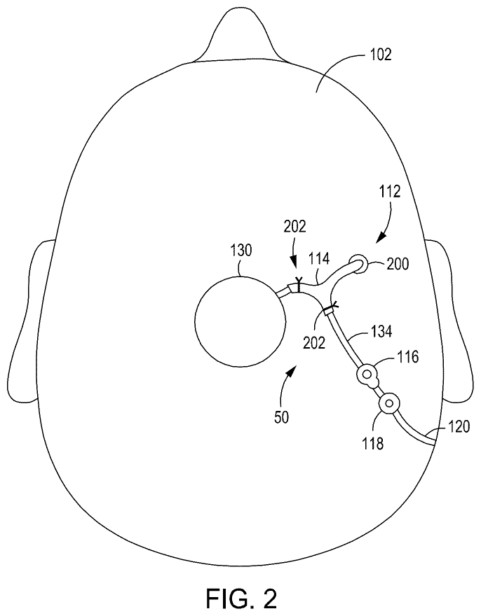

FIG. 2 depicts a top view of the head 102 showing the subgaleal positioning of the artificial fontanelle 130 in accordance with at least one embodiment of the invention. In one embodiment, the artificial fontanelle 130 is positioned centrally, on top of the head 102. The three-way connector 114 is connected between the ventricular catheter 112 (in the burr hole 200), the artificial fontanelle 130, and the coupling catheter 134. In one embodiment, the connector 114 is manufactured of plastic. The catheters, 112, 134 and the artificial fontanelle 130 may be attached to the connector 114 using a ligature 202 formed from a tied suture.

FIG. 3 depicts a horizontal cross-sectional view of a three-way connector 114 of the shunt 50 of FIG. 2 in accordance with at least one embodiment of the invention. FIG. 4 depicts a vertical cross-sectional view of the three-way connector 114 of the shunt 50 of FIG. 2 in accordance with at least one embodiment of the invention. The connector 114 comprises a first port 300, a second port 302 and a third port 304. Ports 300 and 302 have a larger inner diameter than the inner diameter of the third port 304. In one exemplary embodiment, the larger diameter is about 2 mm and the smaller diameter is about 0.5 mm. Optionally, the connector 114 may include a 90-degree bend 400 to position the first port 300 within the burr hole in the skull. In other embodiments, the connector 114 may not have the bend 400 and the ventricular catheter is then curved into the burr hole.

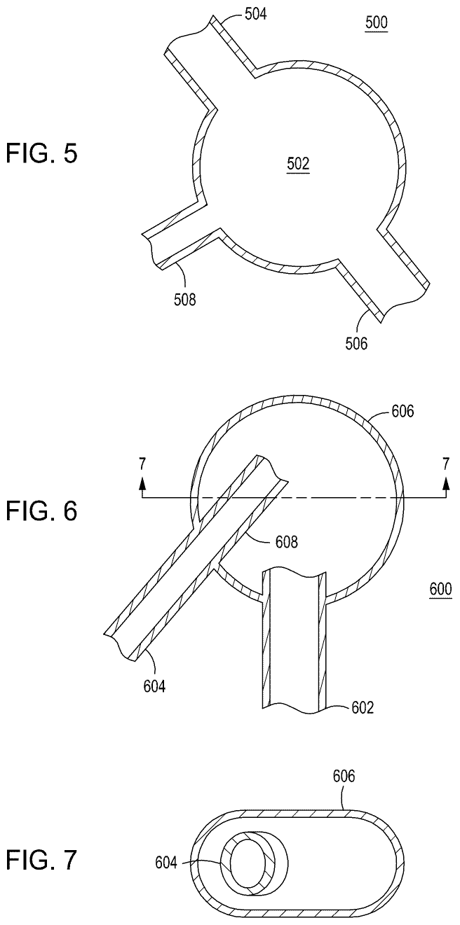

FIG. 5 depicts a horizontal cross-sectional view of a three-way connector 500 with a non-compliant reservoir 502 in accordance with at least one alternative embodiment of the invention. The connector 500 comprises a first port 504, a second port 506 and a third port 508. Ports 504 and 506 have a larger inner diameter than the inner diameter of the third port 508. In one exemplary embodiment, the larger diameter is about 2 mm and the smaller diameter is about 0.5 mm. The non-compliant reservoir 502 may be used for shunt pumping or for tapping CSF from the shunt.

FIG. 6 depicts a horizontal cross-sectional view of an artificial fontanelle 600 in accordance with at least one alternative embodiment of the invention. FIG. 7 depicts a vertical cross-sectional view of the artificial fontanelle 600 along line 7-7 in FIG. 6 in accordance with at least one alternative embodiment of the invention. In the prior embodiments, the artificial fontanelle 130 had a single port and was coupled to a three-way connector 114. In this embodiment, the artificial fontanelle 600 comprises a first port 602 and a second port 604 where the first port 602 is connected to the ventricular catheter (112 in FIG. 1) and the second port 604 is connected to coupler catheter 134. The first port 602 has a larger inner diameter than the second port 604 (e.g., respectively about 2 mm and 0.5 mm). The maximum volume of CSF is about 0.8 mL to 5 mL. In one exemplary embodiment, the volume is between about 2 mL and about 5 mL.

In this embodiment, the reservoir 606 is substantially circular and defined by a compliant sac, bladder, balloon or bag formed of at least one flexible plastic membrane wall. The reservoir 606 expands (volumetrically thickens) and contracts (volumetrically thins) as CSF flows into or out of the reservoir 606. A tube 608 defining port 604 terminates deep into the reservoir 606 to ensure that reservoir does not collapse or twist when empty and cause CSF to flow directly from port 602 into lumen 608 without first filling the reservoir 606.

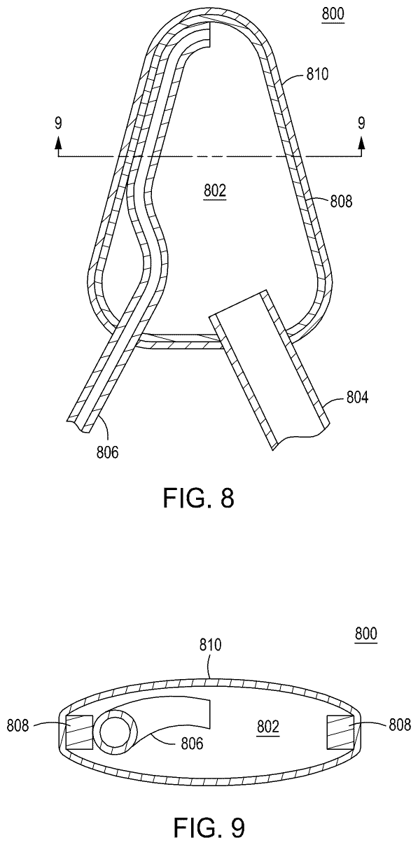

FIG. 8 depicts a horizontal cross-sectional view of an artificial fontanelle 800 in accordance with at least one alternative embodiment of the invention. FIG. 9 depicts a vertical cross-sectional view of the artificial fontanelle 800 along line 9-9 in FIG. 8 in accordance with at least one alternative embodiment of the invention. In this embodiment, the artificial fontanelle 800 comprises a reservoir 802, a first port 804 coupled to the ventricular catheter (112 of FIG. 1) and a tube 806. The tube 806 may be used as the coupler catheter (134 in FIG. 1) or may be connected to the coupler catheter. As with other embodiments, port 804 has a larger inner diameter than the tube 806 to promote CSF flow between the ventricle and artificial fontanelle 800.

In this embodiment, the reservoir 802 comprises a frame 808 that defines the shape of the reservoir 802. The compliant sac, bladder, balloon or bag 810 is formed or positioned over the frame 808 (i.e., a single wall surrounding the frame). In other embodiments, the frame 808 may have a top and bottom membrane wall attached to the frame 808. Although the frame 808 defines the depicted reservoir 802 as pair-shaped, the reservoir may be any shape, e.g., circular, oval, rounded square, and the like. The reservoir 802 expands (volumetrically thickens) and contracts (volumetrically thins) as CSF flows into or out of the reservoir 802 (i.e., the membrane bellows in or out as fluid flows in or out of the reservoir 802). The tube 806 terminates deep into the reservoir 802 to ensure that reservoir does not collapse when empty and cause CSF to flow directly from port 804 into tube 806 without first filling the reservoir 802. Additionally, having a long tube 806 increases the flow resistivity of the tube 806 and promote CSF flow between the artificial fontanelle 800 and the ventricle via the first port 804.

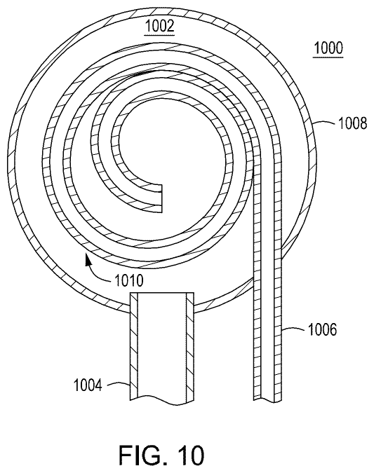

FIG. 10 depicts a horizontal cross-sectional view of an artificial fontanelle 100 in accordance with at least one alternative embodiment of the invention. In this embodiment, the artificial fontanelle 1000 comprises a reservoir 1002, a first port 1004 coupled to the ventricular catheter (112 of FIG. 1) and a tube 1006. The tube 1006 may be used as the coupler catheter (134 in FIG. 1) or may be connected to the coupler catheter. As with other embodiments, port 1004 has a larger inner diameter than the tube 1006 to promote CSF flow between the ventricle and artificial fontanelle 1000.

In this embodiment, the reservoir 1002 may comprise a frame 1008 that defines the shape of the reservoir 1002 or it may be frameless. The tube 1006 forms a spiral 1010 to increase the length of the tube 1006 and further increase its resistance to CSF flow compared to the flow resistance through the port 1004. The spiral 1010 also ensures that reservoir does not collapse when empty and cause CSF to flow directly from port 1004 into lumen 1006 without first filling the reservoir 1002.

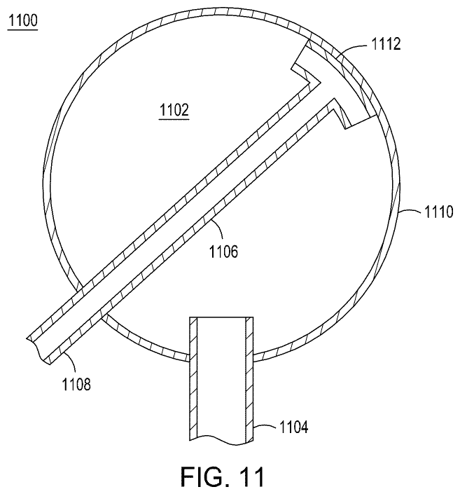

FIG. 11 depicts a vertical cross-sectional view of an artificial fontanelle 1100 in accordance with at least one alternative embodiment of the invention. In this embodiment, the artificial fontanelle 1100 comprises a reservoir 1102, a first port 1104 coupled to the ventricular catheter (112 of FIG. 1) and a rigid tube 1106. The tube 1106 is connected to the coupler catheter (134 in FIG. 1) at port 1108. As with other embodiments, port 1104 has a larger inner diameter than the tube 1106 to promote CSF flow between the ventricle and artificial fontanelle 1100.

In this embodiment, the reservoir 1102 may comprise a frame 1110 that defines the shape of the reservoir 1102 or it may be frameless. The lumen 1106 extends across the reservoir 1102 and comprises a T-shaped port 1112 at its distal end. The length of the lumen 1106 and the T-shaped port 1112 increase the resistance to CSF flow compared to the flow resistance through the port 1104. The rigid lumen 1106 ensures that the reservoir 1102 does not collapse when empty and cause CSF to flow directly from port 1104 into lumen 1106 without first filling the reservoir 1102.

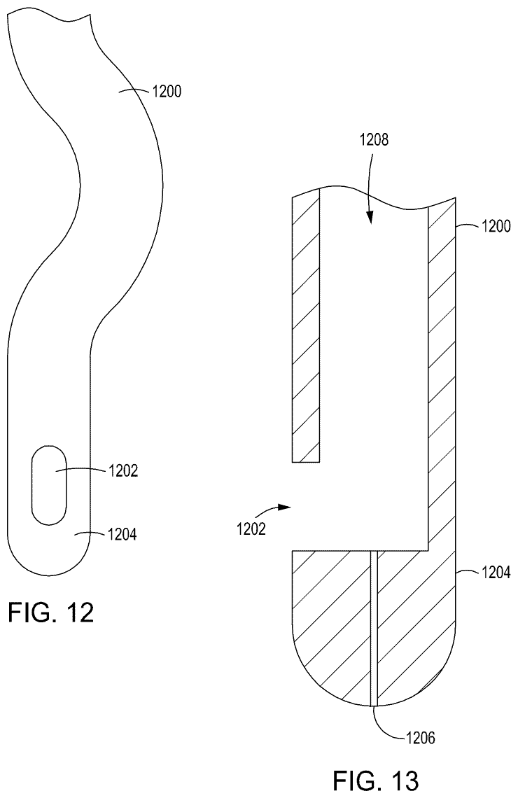

FIG. 12 depicts a perspective view of a ventricular catheter 1200 for use with an artificial fontanelle (e.g., artificial fontanelles 130, 500, 600, 800, 1000, 1100) in accordance with at least one embodiment of the invention. FIG. 13 depicts a vertical cross-sectional view of the ventricular catheter 1200 of FIG. 12 in accordance with at least one embodiment of the invention. To facilitate the free flow of CSF between the ventricle and the artificial fontanelle, the catheter 1200 has a large inner diameter (e.g., about 2 mm) and comprises a single large aperture 1202 located near the distal end 1204. The distal end 1204 is closed. However, in some alternative embodiments, the distal end may comprise a slit 1206. The slit 1206 enables a fiber optic cable or other catheter placement device (not shown) to be inserted into the tube 1208 and through the slit 1206. The closed end 1204 is thick enough to seal the slit 1206 when the fiber optic cable or other catheter placement device is removed. The slit is small enough not to allow proteins or a portion of the choroid plexus to enter the slit and cause tethering of the catheter 1200. The aperture 1202 is generally positioned in the ventricle facing away from the choroid plexus. In this manner, CSF will freely flow in and out of the large lumen tube 1208 and aperture 1202 and not have a portion of the choroid plexus caught in the aperture 1202. In one embodiment, the aperture 1202 is about 1.8 mm wide by 2.4 mm long. The aperture size may be larger or smaller in other embodiments.

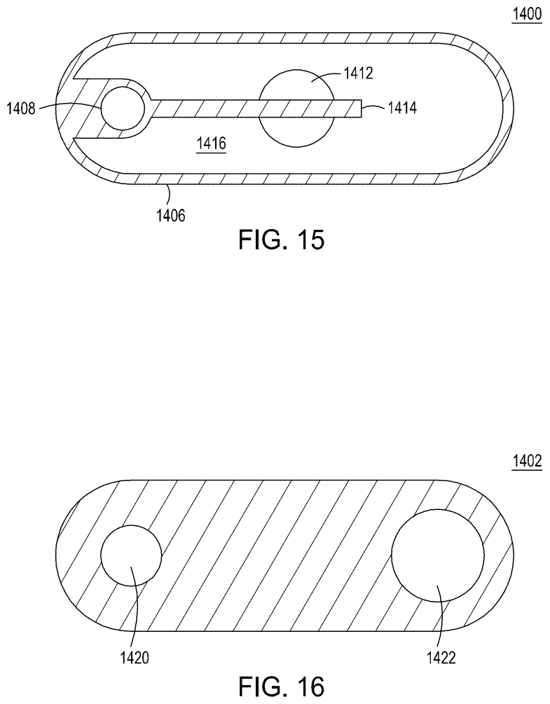

FIG. 14 depicts a vertical cross-sectional view of a vertical-horizontal sensing ball valve 1400 in accordance with at least one embodiment of the invention. FIG. 15 depicts a horizontal cross-sectional view of the vertical-horizontal sensing ball valve 1400 along line 15-15 of FIG. 14 in accordance with at least one embodiment of the invention. FIG. 16 depicts a horizontal cross-sectional view of a dual lumen catheter 1402 along line 16-16 of FIG. 14 in accordance with at least one embodiment of the invention. In one embodiment, the optional vertical-horizontal sensing ball valve 1400 is used to control the flow of CSF from the artificial fontanelle to the peritoneal cavity. The optional vertical-horizontal sensing ball valve 1400, when used, may be positioned in the distal catheter 120 at the location of the valve 116 and ant-siphon device 118. In operation, the ball valve 1400 facilitates a higher flow of CSF when a person is prone (horizontal) than when a person is sitting or standing (vertical).

The vertical-horizontal sensing ball valve 1400 comprises an input port 1404, a ball chamber 1406, a first outlet port 1408, a second outlet port 1410, and a ball 1412. The input port 1404 is fluidly coupled to the first outlet port 1408. The first outlet port 1408 has a smaller inner diameter than the inner diameter of the second outlet port 1410. The ball chamber 1406 comprises a ball stop 1414 extending into the ball cavity 1416 defined by the ball chamber 1406. The ball stop 1414 limits the travel distance (arrow 1418) of the ball 1412 and ensures the ball cannot close the input port 1404. When a person is horizontal, the ball 1412 opens the cavity 1416 to the second outlet port 1410 by having the ball move from position A (POS A) to position B (POS B). When the person is vertical, the ball 1412 moves to position A and blocks the CSF flow from the cavity 1416 to the second outlet port 1410. In this manner, there is a larger amount of CSF flow when a person is horizontal than when the person is vertical.

The ball valve 1400 has the first and second outlet ports 1408 and 1410 coupled to a dual lumen catheter 1402. Specifically, the first outlet port 1408 is inserted in a first lumen 1420 and the second outlet port 1410 is inserted in a second lumen 1422. The first lumen 1420 has a smaller inner diameter than the second lumen 1422. In one embodiment, the first lumen 1420 has an inner diameter of about 0.5 mm and the second lumen 1422 has an inner diameter of about 0.65 mm. In other embodiments the lumen sizes may be larger or smaller.

Here multiple examples have been given to illustrate various features and are not intended to be so limiting. Any one or more of the features may not be limited to the particular examples presented herein, regardless of any order, combination, or connections described. In fact, it should be understood that any combination of the features and/or elements described by way of example above are contemplated, including any variation or modification which is not enumerated, but capable of achieving the same. Unless otherwise stated, any one or more of the features may be combined in any order.

As above, figures are presented herein for illustrative purposes and are not meant to impose any structural limitations, unless otherwise specified. Various modifications to any of the structures shown in the figures are contemplated to be within the scope of the invention presented herein. The invention is not intended to be limited to any scope of claim language.

Where “coupling” or “connection” is used, unless otherwise specified, no limitation is implied that the coupling or connection be restricted to a direct physical coupling or connection.

Where conditional language is used, including, but not limited to, “can,” “could,” “may” or “might,” it should be understood that the associated features or elements are not required. As such, where conditional language is used, the elements and/or features should be understood as being optionally present in at least some examples, and not necessarily conditioned upon anything, unless otherwise specified.

Where lists are enumerated in the alternative or conjunctive (e.g., one or more of A, B, and/or C), unless stated otherwise, it is understood to include one or more of each element, including any one or more combinations of any number of the enumerated elements (e.g. A, AB, AC, ABC, ABB, etc.). When “and/or” is used, it should be understood that the elements may be joined in the alternative or conjunctive.

While the foregoing is directed to embodiments of the present invention, other and further embodiments of the invention may be devised without departing from the basic scope thereof, and the scope thereof is determined by the claims that follow.

1. Apparatus for shunting cerebrospinal fluid (CSF) comprising:
a ventricular catheter having a proximal end adapted to be positioned in a ventricle of a brain; and
a three-way connector having a first port coupled to the ventricular catheter, a second port coupled to an artificial fontanelle, and a third port coupled to a valve, and a peritoneal catheter; and wherein the first port and second port have a larger inner diameter than the third port.

2. The apparatus of claim 1 wherein the artificial fontanelle comprises at least one flexible wall defining a volume adapted for containing CSF.

3. The apparatus of claim 2 wherein the artificial fontanelle is adapted for placement on top of a person's head and, upon the person moving, CSF is caused to bidirectionally flow between the ventricle and the artificial fontanelle.

4. The apparatus of claim 1 wherein the ventricular catheter comprises a single aperture located near a distal end of the ventricular catheter.

5. The apparatus of claim 4 wherein aperture is 1.8 mm by 2.4 mm.

6. The apparatus of claim 4 wherein the distal end comprises a slit.

7. The apparatus of claim 1 wherein the artificial fontanelle comprises a frame to support the at least one flexible wall.

8. The apparatus of claim 1 wherein the valve is a vertical-horizontal sensing ball valve.

9. The apparatus of claim 8 wherein the vertical-horizontal sensing ball valve comprises an input port, a first outlet port, and a second outlet port, where each port is coupled to a cavity and the cavity contains a ball that selectively closes or opens the second port, and where the first and second ports are coupled to a double lumen peritoneal catheter.

10. Apparatus for shunting cerebrospinal fluid (CSF) comprising:
a ventricular catheter having a proximal end adapted to be positioned in a ventricle of a brain;
an artificial fontanelle coupled to the ventricular catheter; and
a peritoneal catheter coupled to the artificial fontanelle.

11. The apparatus of claim 10 wherein the artificial fontanelle comprises a first port coupled to the ventricular catheter and a second port coupled to the peritoneal catheter, where the first port has a larger inner diameter than the second port.

12. The apparatus of claim 10 wherein the artificial fontanelle comprises at least one flexible wall defining a volume adapted for containing CSF.

13. The apparatus of claim 12 wherein the artificial fontanelle is adapted for placement on top of a person's head and, upon the person moving, CSF is caused to bidirectionally flow between the ventricle and the artificial fontanelle.

14. The apparatus of claim 10 wherein the ventricular catheter comprises a single aperture located near a distal end of the ventricular catheter.

15. The apparatus of claim 14 wherein aperture is 1.8 mm by 2.4 mm.

16. The apparatus of claim 14 wherein the distal end comprises a slit.

17. The apparatus of claim 10 wherein the artificial fontanelle comprises a frame to support the at least one flexible wall.

18. The apparatus of claim 10 wherein the valve is a vertical-horizontal sensing ball valve.

19. The apparatus of claim 18 wherein the vertical-horizontal sensing ball valve comprises an input port, a first outlet port, and a second outlet port, where each port is coupled to a cavity and the cavity contains a ball that selectively closes or opens the second port, and where the first and second ports are coupled to a double lumen peritoneal catheter.
